(19) United States
(12) Patent Application Publication
(10) Pub. No.: US 2023/0080204 A1
(43) Pub. Date: Mar. 16, 2023

(54) SYSTEMS, DEVICES, AND METHODS TO PREVENT AUTO AND XENO GRAFT FAILURE

(71) Applicant: The Board of Trustees of the Leland Stanford Junior University, Stanford, CA (US)

(72) Inventors: Abhay Bangalore Ramachandra, New Haven, CT (US); Alison Marsden, Stanford, CA (US); Jay Humphrey, New Haven, CT (US); Jack Boyd, Stanford, CA (US); Hanjay Wang, Stanford, CA (US); Michael Paulsen, Stanford, CA (US); Joshua Pickering, Stanford, CA (US); Muhammad Khan, Stanford, CA (US)

(73) Assignee: The Board of Trustees of the Leland Stanford Junior University, Stanford, CA (US)

(21) Appl. No.: 17/961,489

(22) Filed: Oct. 6, 2022

Related U.S. Application Data
(63) Continuation of application No. PCT/US2021/026443, filed on Apr. 8, 2021.
(60) Provisional application No. 63/006,816, filed on Apr. 8, 2020.

(51) Int. Cl. A61F 2/07

(52) U.S. Cl. CPC A61F 2/07; A61F 2240/002; A61F 2210/0076; A61F 2210/0004

(57) ABSTRACT

Adaptive graft assemblies and methods of manufacture and implantation are provided herein. In particular, such grafts can be 3D printed and can be defined as standard designs or patient-specific, external sheaths customized for specific vein graft dimensions following minimally/non-invasive vein mapping and computational modeling. The external sheath may include one or more layers of various biomaterials to produce customized biomechanical properties. The external sheath may be made to elute specific bioactive drugs allowing for pharmacologic prevention of adverse remodeling in addition to mechanical support. These customizable features may be tailored for each patient individually depending on specific medical history, including hypertension, diabetes, smoking history, anatomy or any pertinent patient attribute. These methods protect vascular grafts, specifically venous grafts, from immediate exposure to arterial pressure that can induce adverse remodeling and graft failure, thereby providing a precision medicine solution to cardiovascular bypass surgery.

### CROSS-REFERENCE TO RELATED APPLICATIONS

The present application is a continuation of International Application No. PCT/US2021/026443, filed Apr. 8, 2021, which claims the benefit of priority of U.S. Provisional Application No. 63/006,816 filed Apr. 8, 2020, the entire disclosure of which is incorporated herein by reference.

### STATEMENT OF GOVERNMENT SUPPORT

The invention was made with Government support under contract HL123689 awarded by the National Institutes of Health. The Government has certain rights in the invention.

### FIELD OF THE INVENTION

The present disclosure relates to grafts, in particular, reinforced graft designed to mitigate maladaptation and failure after surgery.

### BACKGROUND

Each year, approximately 400,000 coronary artery bypass grafting (CABG) surgeries, 55,000 arterio-venous fistula creation procedures, and 20,000 peripheral artery bypass surgeries are performed in the United States alone. Vein graft failure rates are as high as 50% at 10 years for CABG vein grafts, 25-55% at 5 years for infragenicular bypass grafts, and the primary failure rate of arterio-venous fistulas is approximately 40% at the end of one year. The cost of fistula failure alone is estimated to be at $1.2B annually, while the cost of CABG vein graft failure is estimated to be even higher. Repeat procedures lead to higher morbidity and mortality, costing the healthcare system an additional several billion dollars and causing the patients a lower quality of life. Graft failure costs the healthcare system billions of dollars per year. Various approaches to improve grafts have been proposed, including those in U.S. Patent Publications: U.S. Pat. Nos. 9,265,632; 9,579,224; 2014/0303715; 2012/0330437; U.S. Pat. Nos. 8,361,101; and 9,517,121, yet currently, these improvements have failed to satisfactorily resolve the considerable problem of graft failures. Therefore, there is need for improved graft structures that mitigate maladaptation after surgery, thereby reducing or avoiding graft failure.

### BRIEF SUMMARY

The present disclosure relates to graft assemblies and associated methods, in particular, grafts with customized and/or adaptive reinforcement that mitigates maladaptation and failure of autologous vascular grafts and xenografts after surgery. In one aspect, the graft systems and methods described herein are advantageous for a variety of clinical applications, in particular, coronary bypass surgery, peripheral arterial disease, and arteriovenous fistula. It is appreciated, however, that these systems and methods could be applied to any graft application in accordance with the concepts described herein.

Vein grafts are ubiquitous in clinical applications including coronary artery bypass, peripheral artery bypass, and arterio-venous fistulas. Vein grafts have a very high failure rate, however, which increases healthcare expenses by billions of dollars per year. The devices and methods proposed herein are designed to prevent failure of vascular grafts in general, and in particular vein grafts. In some embodiments, the methods allow for the production of patient-specific, 3D-printed external sheaths to be custom-made for specific vein graft dimensions by use of vein mapping and/or computational modeling. In some embodiments, the device is manufactured in multiple sizes (e.g., diameters and/or lengths) and therefore may not be patient specific or custom to the individual. In some embodiments, the sheath is a single layer. In other embodiments, the sheath is multiple layers. The sheath can be formed by 3D printing. In some embodiments, the sheath is formed of a polymer that can be elastomeric and/or biodegradable. In some embodiments, the device is formed in a lattice design (e.g. with pores or opening), typically a lattice design that can be 3D printed. In some embodiments, the external sheath includes one or more layers of one or more biomaterials selected or customized to produce biomechanical properties suitable for a given patient. In some embodiments, the external sheath has a braided structure. In some embodiments, the external sheath can include one or more layers that elute specific bioactive drugs to allow for pharmacologic prevention of adverse remodeling in addition to reinforced mechanical support. These customizable features may be tailored for each patient individually depending on their specific medical history, including hypertension, diabetes, smoking history, or any relevant patient attribute. In one aspect, the methods described herein protect vascular grafts, specifically venous grafts, during immediate exposure to arterial pressure that can induce adverse remodeling and graft failure. The outer sheath protects the vein graft from overdistension and deleterious consequences from injury when subjected to arterial pressure after implantation. The approaches described herein allow for graft designs that support a precision medicine solution to cardiovascular bypass surgery.

Current surgical practices do very little to aid in preventing a vascular graft (usually a vein graft) from maladaptation after implantation. Indeed, the common intraoperative practice of distending the graft prior to implantation can even worsen the maladaptive response. Coronary artery bypass grafting (CABG) surgery is performed on nearly half a million patients with multivessel or diffuse coronary artery disease each year in the United States. Surgeons have a choice of arterial or venous grafts, and while arterial grafts offer superior performance, they are limited in availability. Venous grafts, therefore, are used in about 95% of patients who undergo CABG. Venous grafts occlude and fail, however, at a rate of 50% within 5-10 years after surgery, leading to repeat revascularization procedures, myocardial infarction, or death in 30% of patients within 5 years of graft failure. It is well known that adverse remodeling plays a major role in vein graft occlusion. This is due in part to the sudden drastic change in biomechanical loading when the vein is transposed into the arterial circulation as a bypass graft and immediately faces arterial blood pressure. Recent modeling and experimental efforts from our team have demonstrated that a gradual change in mechanical loading on the vein graft may enable more favorable adaptation to arterial pressure, thus minimizing the risk of long term graft failure. Thus, the purpose of the improved graft designs described herein is to mitigate maladaptation and failure of autologous vascular grafts and xenografts after surgery. In some embodiments, the graft design mitigates graft maladaptation by utilizing a custom-fit, non-blood contacting device, made from biodegradable or bioresorbable materials.

To achieve this in practice, the graft assembly can utilize a single or multilayer, patient-specific, 3D-printed sheath composed of biocompatible, biodegradable, elastomeric, and/or drug eluting biomaterials to serve as an external support for prevention of vein graft failure. The sheath can be customized for the patient or can be made in varying sizes and shapes that are then selected for a particular patient. Typically, the external support is non-blood contacting. As described herein, the external support or sheath placed over the graft vessel is referred to as a graft assembly or graft system. The graft systems described herein may be constructed of various biomaterials including hydrogels, the composition of which may be carefully tuned to produce different biomechanical properties depending on the size, location, hemodynamic load, geometry, and cellular composition of the vascular graft. In some embodiments, the sheath is formed of a biodegradable material that is designed to maintain structural integrity for at least a short time after surgery (e.g. 1-6 weeks). In some embodiments, each layer of a multi-layer sheath may be constructed using different biomaterials to produce a graduated degradation. In some embodiments, the external support can be made to elute specific drugs that promote endothelial health, inhibit adverse remodeling processes, reduce exuberant cell proliferation and matrix production, stimulate angiogenesis in the local tissues, or achieve other molecular and cellular benefits.

In some embodiments, the external support has varied biodegradability of sheath layers at different luminal diameters allowing for different stages of restriction from overexpansion, conformability to tortuosity and profile of graft course, and ability to specifically size the sheath for each individual vein graft based on the sheath design and pre-operative imaging. In addition, incorporation of drugs, immune or inflammatory modulators can further aid in the optimal arterialization of venous conduits.

### BRIEF DESCRIPTION OF THE DRAWINGS

FIGS. 1A-1B shows cross-sectional side views of single layer and multi-layer graft assemblies in accordance with some embodiments of the invention.

FIG. 2 shows a manufacturing method of forming an external support utilizing clinical imaging in accordance with some embodiments.

FIG. 3 shows a method of manufacturing an exemplary external support having a braided external support in accordance with some embodiments.

FIG. 4 shows a method of implanting a graft assembly having a braided external support in accordance with some embodiments.

FIGS. 5A-5B shows an external graft support having a lattice design in accordance with some embodiments.

FIGS. 6A-6E show a method of implanting a graft assembly having an external support of a lattice design in accordance with some embodiments.

### DESCRIPTION OF THE INVENTION

Coronary artery bypass grafting (CABG) surgery is performed on nearly half a million patients with multivessel or diffuse coronary artery disease each year in the United States. Surgeons have a choice of arterial or venous grafts, and while arterial grafts offer superior performance, they are limited in availability. Venous grafts, therefore, are used in about 95% of patients who undergo CABG. Venous grafts occlude and fail, however, at a rate of 50% within 5-10 years after surgery, leading to repeat revascularization procedures, myocardial infarction, or death in 30% of patients within 5 years of graft failure. It is well known that adverse remodeling plays a major role in vein graft occlusion. It is believed this is due in part to the sudden drastic change in biomechanical loading when the vein is transposed into the arterial circulation as a bypass graft and immediately faces arterial blood pressure. Recent modeling and experimental efforts from our team and others have demonstrated that a gradual change in mechanical loading applied to the vein graft may enable more favorable adaptation to arterial pressure and flow, thus minimizing the risk of long-term graft failure. To achieve this in practice, the graft assemblies described herein utilize an external support that is provides an adaptive response after surgery in order to mitigate the above-noted problems and avoid graft failure. In other embodiments, the external support can be made in varying sizes, shapes and properties and selected according to a particular patient's anatomy and needs. In some embodiments, the graft assembly utilizes a single layer sheath, which could be 3D printed or designed as a wrap, composed of biocompatible, biodegradable and/or bioresorbable elastomeric biomaterials, with possible drug-eluting capabilities, to serve as an external adventitial support for prevention of vein graft failure. Alternate techniques could include dip coating, electrospinning, extrusion, sheet wrapping and salt-leaching. In some embodiments, the external support is customized or designed to be patient-specific. In some embodiments, a plurality of external sheaths are provided having varying dimensions (e.g. diameters, lengths, shapes) and/or varying characteristics (e.g. strength, durability, biodegradability, drug elusion), from which a clinician can select a suitable external support for a graft assembly in a given patient based on the patient's anatomy or the required needs of a given procedure.

### I. Graft Assemblies

In one aspect, the approaches described herein represents a novel pathway for the construction of cost-effective, patient-specific, biodegradable, external sheaths for vein graft support. Vein geometry may be determined using minimally/non-invasive mapping which is routinely performed for patients prior to cardiovascular bypass surgery. Imaging data, together with image segmentation and anatomic model construction methods, can be used to construct a virtual computer model of the external sheath matched precisely to the design size and geometry of the graft to be used in the bypass surgery. It is understood that the size and geometry of a desired graft may be different from that of the vein in which the graft is mounted, for example, an oversized graft can be used. In some embodiments, each graft constructed in this process would be custom designed for the patient. In some embodiments, the sheath is 3D printed to be available at the time of surgery. Optionally, 3D printing could be integrated into the normal clinical workflow.

Further customization of the sheath for each patient may include the use of different biomaterials in a single or multi-layer sheath design to produce the optimal biomechanical properties (e.g. for hypertensive vs non-hypertensive patients), or loading of the sheath with specific drugs to be eluted depending on the patient's medical history (e.g. for diabetic smokers vs non-diabetic nonsmokers, or any combination of comorbidities). The custom sheath would then be applied and affixed to the vein during the CABG surgery (e.g. affixed by clips, tissue glue, sutures, naturally apposed, “tight fit”, or by any suitable affixation means). Using the custom 3D models of veins, it would also be possible to perform virtual surgery of CABG hemodynamics or virtual remodeling and adaptations in patient-specific models. These novel features support a precision medicine solution to cardiovascular bypass surgery.

In one aspect, the external support is selected or designed to match the properties of the vasculature to which the grafts are mounted. In some embodiments, the structural stiffness (product of material stiffness and thickness) of the external support should match the structural stiffness of the adjacent vasculature. The material stiffness of the material can range from 1 MPa (megapascals) to 10 GPa (gigapascals), which includes the stiffness of bioabsorbable polymers such as PPF (poly(propylene fumurate)), PGS (poly(glycerol sebacate)), PCLA (polycaprolactone-co-lactide), PLA (polylactide), PLLA (poly(l-lactic acid)), PCL (polycaprolactone), and PGA (polyglycolide), polyvinylidene fluoride (PVDF), polyurethane (PU), polypropylene (PP) and PP, poly(ε-caprolactone) (PεCL), or any combination of these materials, which are common polymeric materials used in grafts, and from which some embodiments of the grafts described herein can be formed. In some embodiments, the external support is generally tubular with a length in between 2 cm and 60 cm, typically between 5 cm and 20 cm, such as within 10-12 cm, and a diameter between 0.1 cm and 3 cm, typically between 0.1 cm and 2 cm, such as within 0.2-1 cm, or 0.2-0.5 cm. In some embodiments, the thickness of the external support can range from 0.1-10 mm, typically about 0.2-1 mm. It is appreciated that the external support could be of any suitable dimensions selected to suit a patient's vasculature or the requirements for a particular procedure. The external support can have the size and modulus to maintain a uniform diameter and reduce disturbed flow in the venous segment of the graft to enable favorable hemodynamics.

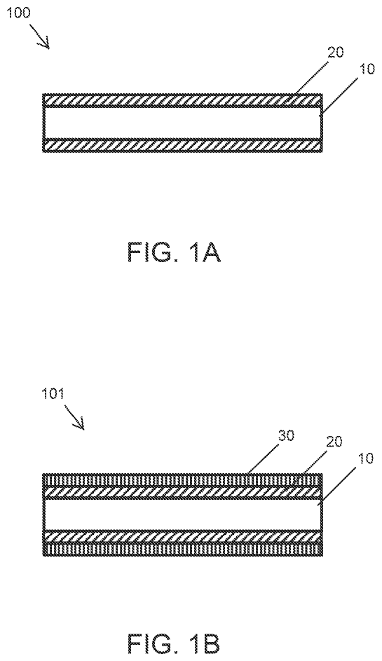

In another aspect, the graft assembly includes an external support with one or more layers, for example as shown in the embodiments in FIGS. 1A-1B. FIG. 1A shows a graft assembly 100 having a vascular graft 10 with an external support 20 comprised of a single layer designed or selected to match design specifications that correspond to properties of the vasculature to which the graft is mounted. The external component 20 can be formed of materials selected or customized, individually or in combination, to match the properties of the natural vasculature or design specifications. In some embodiments, the external support comprises biodegradable material so as to provide further reinforcement immediately after surgery and for a short time thereafter (e.g. 1-6 weeks after), and later dissolve when additional reinforcement is no longer needed, thereby providing properties closer to existing vasculature. In some embodiments, this short period of time thereafter can be any of: 1, 2, 3, 4, 5 or 6 weeks after surgery. In some embodiments, the external support is formed by braiding a material, such as a biodegradable wires, sheets, or mesh, over the graft tube.

FIG. 1B shows a graft assembly 101 having a graft tube external support 10 comprised of two layers 20, 30. The layers can be formed of the same material or different materials having differing properties. In some embodiments, the layers provide differing functions, for example, increased stiffness and drug elution. For example, layer 20 can provide reinforcement against distension and layer 30 can provide drug elution. In some embodiments, one or more of the layers are biodegradable, while others are not biodegradable, in order to provide variability in properties. In some embodiment, multiple layers having differing rates of biodegradability are used to provide design properties that change or adapt over time, thereby mitigating maladaptation and failure after surgery. It is appreciated that the layers and their associated functions could be arranged in any number of ways. Further, it is appreciated that the multi-layer graft assembly is not limited to two layers and can encompass any number of layers desired.

In some embodiments, the number of layers on the external support ranges from one or more layers, typically one to three layers. However, it is appreciated that any number of multiple layers could be used, particularly with microscale and nanoscale manufacturing methods. In addition to providing structural reinforcement, one or more of the layers can be configured to provide a specific function, for example, any of the following or any combination thereof:
Biodegradability with time and maintain mechanical integrity for a set period of time, for example, any of hours, days, weeks, months (e.g. no less than a short period of time, typically within 1-6 weeks, after surgery)
Incorporation of drugs, including macrophage and TGFβ inhibitors, other anti-inflammatory and immune modulators, to prevent excessive inflammation and enable neo-tissue development. The drugs prevent formation of neointimal lesions, inflammatory collagen, and atherosclerotic lesions. The drug-release capabilities could last up to 12 months.
Oversized layer, with respect to vessel diameter at a given pressure (e.g. 10 mmHg) to prevent compressive stress on the native tissue or the neo-tissue
Construction of multi-layered design to match the desired structural stiffness
Sheath design can be configured to provide ease in handling surgically with minimal damage to the sheath or native tissue wall (e.g. resilient lattice design that facilitates handling) and/or to provide ease of integration, without disruption, into the clinical workflow in a CABG surgery (e.g. provided ready for insertion into the workflow without requiring extensive modification of the sheath or extensive modification of clinical workflow)

In yet another aspect, the one or more layers of the external support can be constructed by various manufacturing approaches. In some embodiments, the external support is formed by a braided or wrapped design, as described in the example below. In some embodiments, one or more layers are constructed by an additive manufacturing method, such as 3D printing. Suitable additive manufacturing apparatus and methods on which objects can be produced include bottom-up and top-down additive manufacturing methods and apparatus, as known and described in, for example, U.S. Pat. No. 5,236,637 to Hull, U.S. Pat. Nos. 5,391,072 and 5,529,473 to Lawton, U.S. Pat. No. 7,438,846 to John, U.S. Pat. No. 7,892,474 to Shkolnik, U.S. Pat. No. 8,110,135 to El-Siblani, U.S. Patent Application Publication No. 2013/0292862 to Joyce, and U.S. Patent Application Publication No. 2013/0295212 to Chen et al. In some embodiments, the additive manufacturing step is carried out by one of the family of bottom-up methods sometimes referred to as continuous liquid interface production (CLIP). CLIP is known and described in, for example, U.S. Pat. Nos. 9,211,678; 9,205,601; 9,216,546; and others; in J. Tumbleston et al., Continuous liquid interface production of 3D Objects, Science 347, 1349-1352 (2015); and in R. Janusziewcz et al., Layerless fabrication with continuous liquid interface production, Proc. Natl. Acad. Sci. USA 113, 11703-11708 (2016). Other techniques include: stereolithography (SLA) and digital light processing (DLP). Both are based on photopolymerization or photo-cross-linking processes that remain the fastest 3D printing methods and exhibit higher resolution compared to other techniques, as they are only limited by the light source and pixilation (resolution) of the printer. These and other such techniques can be further understood by referring to U.S. Pat. No. 10,465,044; EP Patent 3063205B1; and PCT Publication No. WO2020/014699A1. These are examples of manufacturing technologies that may be used, however, it is appreciated that formation of the external support and graft assembly is not limited to these methods.

### II. Methods

### A. Method of Manufacturing Customized Graft Assembly

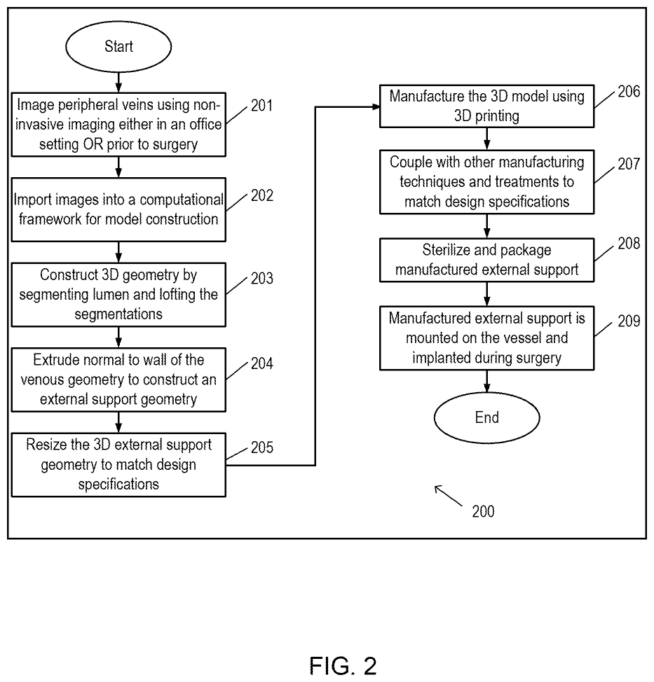

In some embodiments, the design of the external support can be customized for individual patients via image-based modeling. FIG. 2 shows an exemplary manufacturing method 200 of forming an external support by utilizing clinical imaging. Method 200 can include the following steps:

(Step 201) Image the peripheral veins using non-invasive clinical imaging, which can be performed in an office setting or in the operating room prior to surgery. In some embodiments, the imaging modality is ultrasound. Alternative imaging modalities can include CT scan (computerized tomography scan) and MRI (magnetic resonance imaging).

(Step 202) Import the medical images into a computational software framework. In some embodiments, the intensity of the images can be adjusted and images can be processed with filters.

(Step 203) Construct 3D geometry by segmenting the lumen and lofting the segmentations. The computational framework allows for segmentation of the lumen, using 2D segmentation with lofting, 3D segmentation, or machine learning methods, resulting in construction of a 3D model of the vascular geometry. The framework also allows for manipulation of the 3D model.

(Step 204) The external support geometry is constructed by extruding normal to the venous wall surface. Manipulation or optimization, via computer aided design or formal optimization methods, of the 3D external support model would be performed such that the design specifications of the external support are satisfied. Design specifications could include sizing, thickness, length and layers in the graft, and additional material at the ends to allow for or aid the anastomosis.

(Step 205) Resize the 3D external support geometry to match design specifications.

(Step 206) Manufacture the external support using desired manufacturing technique or a hybrid of techniques. In some embodiments, the external support is formed by 3D printing.

(Step 207) Optionally, couple with other manufacturing techniques and treatments to match design specifications. 3D printing can be further coupled with alternate manufacturing techniques and additional treatments to match design specifications. Alternate techniques could include dip coating, electrospinning, extrusion, sheet wrapping and salt-leaching. Additional treatments could include pharmacological seeding and nano-particle embedding.

(Step 208) Sterilize and package the external support for use in the operating room.

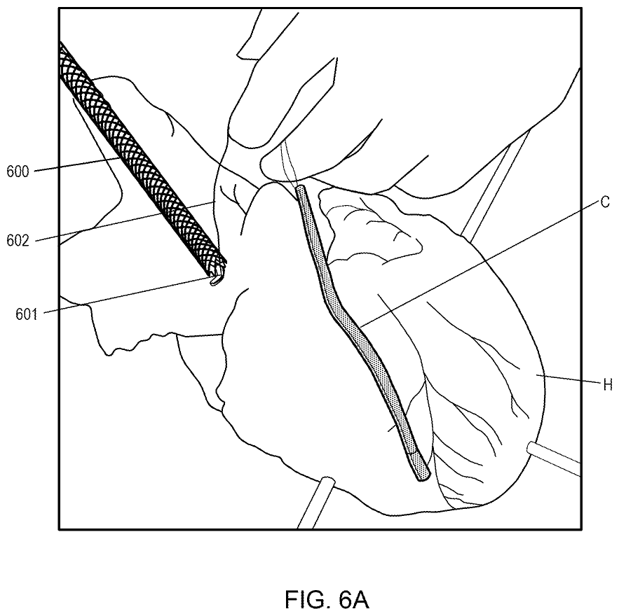

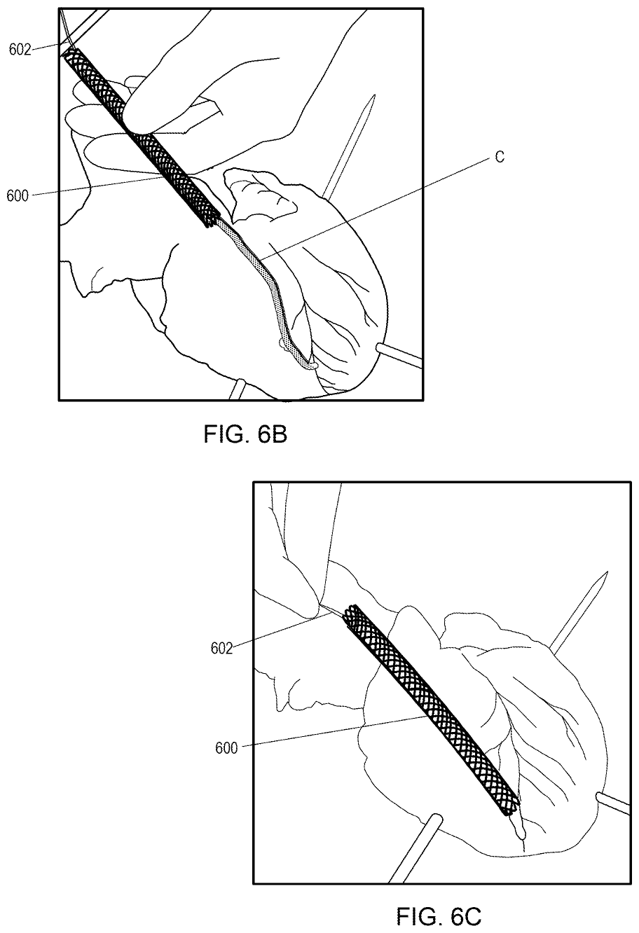

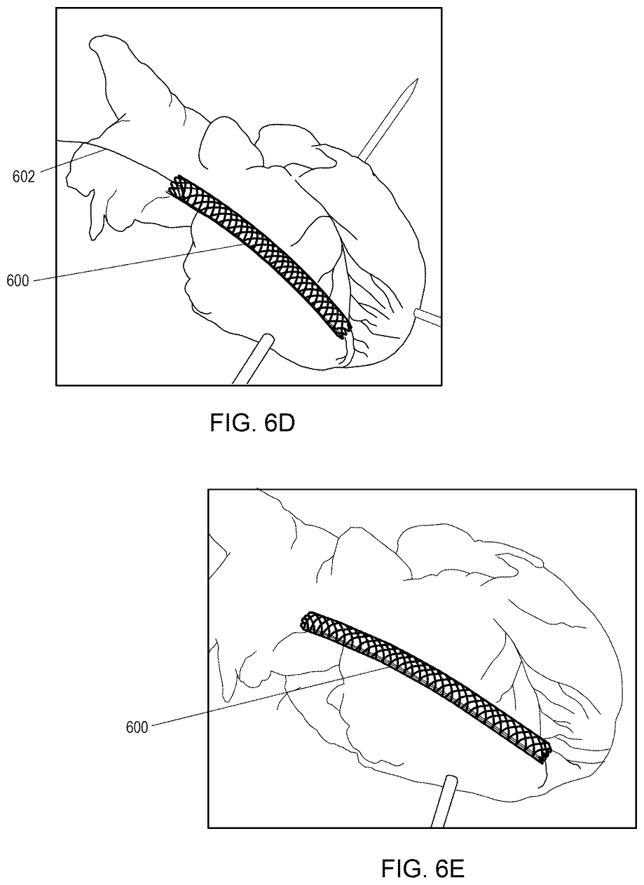

(Step 209) Manufactured external support is mounted on the graft vessel and implanted during surgery. Examples of this step are shown in FIGS. 6A-6B.

It is appreciated that the above method is exemplary and can be modified to exclude or modify one or more steps noted above, or to include additional steps, as desired. While this method shows a particular modeling approach, it is appreciated that the 3D geometry could be performed by any suitable modeling approach. Additionally, it is appreciated that various other manufacturing processes and techniques can be used in the alternative or in addition to those described above.

### B. Method to Manufacture a Braided External Support

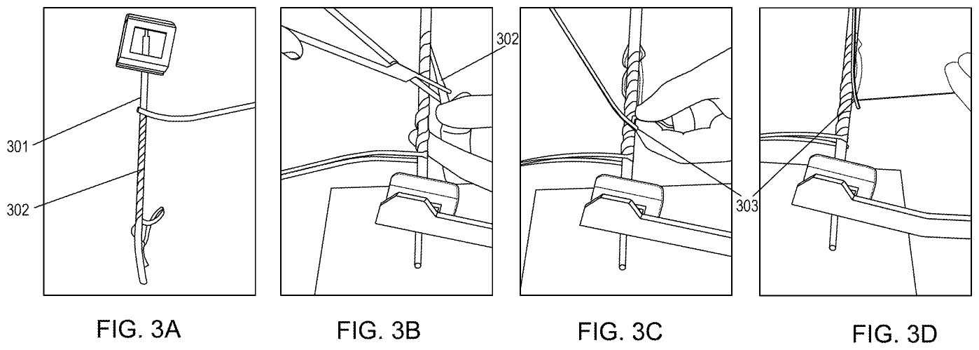

In addition to 3D printing, described above, some embodiments include a braided design that includes multiple layers. FIG. 3 shows a method of manufacturing a braided external support that can include the following steps: Step A shows a customized jig that includes a sterile vice, mandrel 301, and strips of biodegradable mesh 302. In step B, the strips of mesh are braided on the mandrel with pitch and layers to match design specification. In steps C and D, the braided support is then sutured 303 at multiple locations along the length to maintain form and structural integrity. It is appreciated that this is but one approach to forming an external support to create a custom or adaptive graft external support and that alternative and/or additional processes could be utilized.

### C. Method of Loading and Application of Graft Assembly to the Vein

The invention also includes methods of loading and application of the adaptive graft on the vein. For example, using the 3D printed design or the braided design, the sheath would be loaded on the vein. The method can also include the following considerations:

A. Expansion of the sheath diameter using a support device, such as a speculum or a rigid tube with one end narrowed, to enable loading the sheath onto the vein without damage.

B. Alternatively, or in addition, a temporary possibly disposable conduit can be inserted within the sheath to facilitate loading the vein into the sheath.

C. Extensions, such as a skirt or flared sections, added to the ends of the sheath allow for flexible anastomosis to the native tissue.

D. Pressurization of the vein (e.g. to physiologic values of 5-10 mmHg) can be used for size selection of the sheath to maintain target sizes.

E. Axially compressing the sheath allows for greater internal diameter for ease of loading.

F. Axially stretching the sheath adjusts internal diameter to appropriate fit.

### D. Methods to Implant a Braided External Support

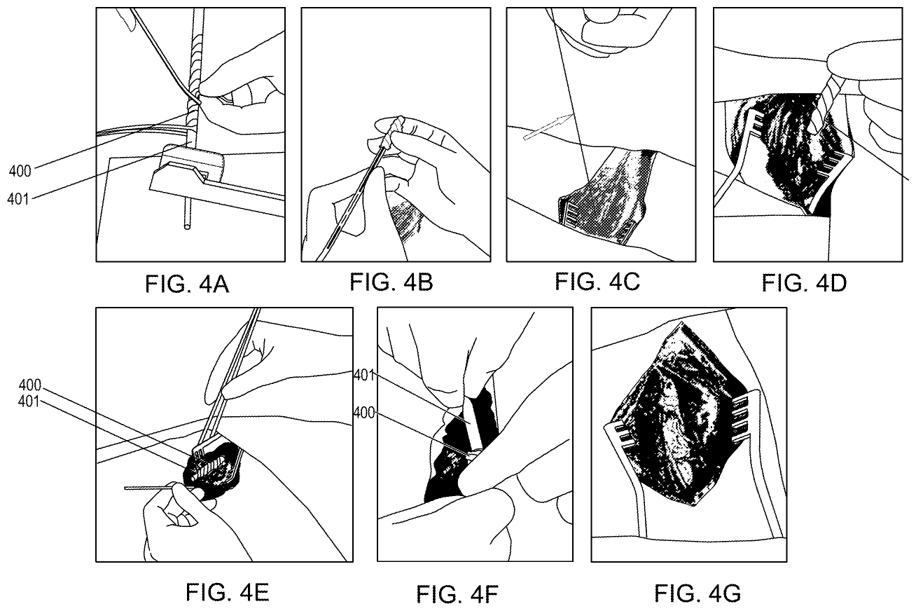

The graft assemblies can be mounted to the vasculature in a conventional manner, or can be implanted in accordance with specialized implantation methods. FIG. 4 shows an exemplary method of implanting a braided external support that includes the following steps:

In Step A, the external support 400 is formed in advance or in the operating room on an internal tube 401. In this example, the support 400 can be formed by braiding (e.g. braided wires, sheets or strips) of biodegradable, as described above. The external support is intermittently stitched, sutured, glued or stapled along its length to improve structural integrity and maintain form. The internal tube is kept within the device until step F. In some embodiments, this process only takes about 10-20 minutes of construction time when performed manually.

In Step B, the braided external support is sized such that it is slightly oversized compared to the excised vein, at 10 mmHg, along both circumferential and axial directions.

In Step C, the proximal anastomosis is created, and hemostasis is confirmed. A guide-suture (white arrow) is sutured to the distal end of the venous perivascular tissue to mount the external support.

In Steps D and E, the external support is slid onto the guide-suture and moved past the proximal anastomosis.

In Step F, the internal tube 401 is pulled out and sizing of the external support is preliminarily verified.

In Step G, the distal anastomosis is created, the system is de-aired, and hemostasis is confirmed. The external support is stretched into its final conformation to cover the distal anastomosis, and the clamps are released, filling the sheathed vein graft. Apposition of the external support with the vein graft is assessed, and the reinforcement layers (e.g., braids) on the external support are adjusted to cover the full length and surface area of the vein. The proximal and distal ends of the external support are secured to the surrounding tissues if necessary.

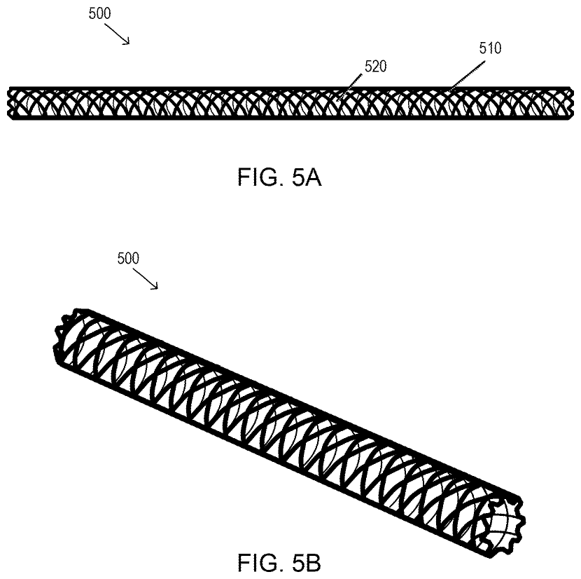

FIGS. 5A-5B depict another embodiment of an external support 500 for a graft system, as described previously. FIG. 5A shows a side view of external support 500 and FIG. 5B shows a perspective view. In this example, external support 500 is a single-layer tubular support 510 formed of a suitable material (e.g. polymer, biodegradable material) and is defined in a lattice work having openings 520 (e.g. pores or interstitial spaces) within the lattice design. In some embodiments, this design has sufficient strength so that the external support can be deployed during the surgical procedure without a removable internal support tube, which allows for greater ease in handling without damage to the support wall or the native tissue and better integration into the clinical workflow.

FIGS. 6A-6D show implantation of the external support 600 of the same design as that in FIGS. 5A-5B. As shown in FIG. 6A, external support 600 can initially be handled during the surgical procedure by a support rod 601 extending therethrough. A guide-suture 602 is attached to the cardiac vessel C (e.g. vein of an anastomosis) of the heart H. The guide-suture 602 can be fed through the external support 600, for example, by use of the internal rod 601 which is withdrawn through support 600. As shown in FIG. 6B, the clinician can then advance the external support 600 over the guide-wire 602 and onto the cardiac vessel C. The clinician continues to advance the external support 600 until fully positioned at the desired location on the cardiac vessel, as shown in FIG. 6C. As shown in FIG. 6D, the guide-suture 602 can be removed/withdrawn. As shown in FIG. 6E, the external support 600 can be secured in place, for example by dissolvable sutures, to ensure the external support 600 remains in place providing reinforcement of the cardiac vessel for at least a short time period after the surgical procedure, as described herein.

In the foregoing specification, the invention is described with reference to specific embodiments thereof, but those skilled in the art will recognize that the invention is not limited thereto. Various features, embodiments and aspects of the above-described invention can be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art. Each of the references cited herein are incorporated herein by reference for all purposes.

1. An external support for a graft assembly, the external support comprising:
an external support tube configured to extend along the outside of a graft allowing passage of blood therethrough; and
a lumen extending through the external support that is sized to facilitate placement of the external support tube over the graft,
wherein the external support body has dimensions and characteristics that are selected or designed to match correspond to the graft or a specific vasculature to which the graft is mounted so as to mechanically reinforce the graft for at least a period of time after the surgical procedure.

2. The external support of claim 1 wherein the external support comprises a single layer of a 3D printed design.

3. The external support of claim 2 wherein the 3D printed design is a lattice design having a plurality of opening therein.

4. The external support of claim 1 wherein the external support is of sufficient strength to be handled during a surgical procedure without an internal tubular support disposed within.

5. The external support of claim 1 wherein the external support is formed of a biodegradable material that maintains structural integrity for at least the period of time after the surgical procedure.

6. The external support of claim 1 wherein the period of time after the surgical procedure is between 1-6 weeks.

7. The external support of claim 1 wherein the external support comprises one or more layers of differing properties, wherein the differing properties include any of biodegradability, bioresorption, biointegration, porosity, stiffness or any combination thereof.

8. The external support of claim 1 wherein the external support comprises braided pieces of biodegradable material.

9. The external support of claim 1 wherein the external support is designed with a structural stiffness that matches or exceeds the specific vasculature to which the graft is mounted.

10. A method of forming a graft assembly, the method comprising:
imaging candidate peripheral veins using non-invasive/minimally imaging in an area where the graft assembly is to be implanted, wherein the graft assembly includes a graft and an external support;
constructing a 3D model of a lumen of the graft and the external support of the graft assembly to be implanted;
adapting the 3D model to match design specifications that are either predefined or determined from the imaging of peripheral veins; and
forming the external support of the graft assembly based on the adapted 3D model.

11. The method of claim 10 wherein forming the external support comprises braiding pieces of biodegradable material to match design specifications.

12. The method of claim 10 wherein the design specifications comprise any of: structural stiffness, thickness of the external support, diameter, or any combination thereof.

13. The method of claim 10 wherein the external support comprises one or more layers.

14. The method of claim 10 wherein forming the graft assembly comprises 3D printing.

15. The method of claim 10 wherein forming the graft assembly utilizes one or more manufacturing processes to match the graft assembly with design specifications, wherein the additional process include any of: dip coating, electrospinning, extrusion, sheet wrapping, salt-leaching or any combination thereof.

16. A method of forming an external support for a graft assembly to provide reinforcement of a graft vessel, the method comprising:
providing 3D design for an external support having a tubular shape having dimensions suitable for placement over the graft vessel, wherein the design is standard or customized for a particular patient; and
forming the external support by 3D printing with a material having suitable characteristics for reinforcing the graft vessel, wherein the material is selected or customized to maintain structural integrity for at least a period of time after the grafting procedure.

17. The method of claim 16, wherein the 3D design is a lattice having a plurality of openings therein.

18. The method of claim 16, wherein the 3D design is a single layer.

19. The method of claim 16, wherein the material is biodegradable and the period of time is within 1-6 weeks.

20. A method of implanting a graft assembly, the method comprising:
providing the graft assembly customized to match or exceed design specifications corresponding to the vasculature to which the graft assembly is to be mounted along a location of an excised vein between a proximal and distal anastomosis, wherein the graft assembly includes a graft and external support, wherein the graft assembly is sized so as to be slightly oversized compared to the excised vein along both circumferential and axial directions;
creating the proximal anastomosis and then affixing a guide-suture to a distal end of the venous perivascular tissue to mount the external support to the vasculature;
sliding the external support onto the guide-suture and moving the external support past the proximal anastomosis; and
creating the distal anastomosis, positioning the external support into a final conformation position covering the distal anastomosis, releasing any clamps restricting blood flow, and checking for apposition of the external support.

21-30. (canceled)
